(19) United States
(12) Patent Application Publication
(10) Pub. No.: US 2002/0045168 A1
(43) Pub. Date: Apr. 18, 2002

(54) Computer based modeling system

(76) Inventors: Mohammad Afshar, Cambridge (GB); Stephen Morley, Cambridge (GB)

Correspondence Address: Kathleen M. Williams, Ph.D., J.D., PALMER AND DODGE, LLP, One Beacon Street, Boston, MA, 02108, US

(21) Appl. No.: 09/747,715

(22) Filed: Dec. 22, 2000

Related U.S. Application Data
(60) Provisional application No. 60/226,782, filed on Aug. 21, 2000.

(51) Int. Cl. C12Q 1/68; G06F 19/00; G01N 33/48; G01N 33/50

(52) U.S. Cl. 435/6; 702/20

(57) ABSTRACT

A method for defining a binding site in a biological macromolecule based on a two-sphere grid and a method for determining the free energy of a ligand:RNA structure based on pseudo-energy values. These methods can be use in docking and also in high-throughput in silico screening of ligand libraries against an RNA structure of interest.

### RELATED APPLICATIONS

This application claims priority under 35 U.S.C. § 119(e) to U.S. Provisional Application Ser. No. 60/226,782, filed Aug. 21, 2000, the entirety of which is incorporated herein by reference.

### FIELD OF THE INVENTION

The invention is in the field of computer-based biomolecular modelling. In particular, the invention relates to the in silico docking of a ligand with a biological receptor, such as the docking of a ligand with RNA and the formation of RNA-ligand complexes. More particularly, the invention provides a way to predict the binding mode and affinity of a ligand for its receptor by starting with a two-dimensional (2D) structure for the ligand and a three-dimensional (3D) stricture for the receptor.

### BACKGROUND

Computer-based molecular docking techniques are widely used for modelling the interaction between a ligand and a biological receptor (e.g., Lengauer and Rarey, Curr. Opin. Struct. Biol. 5: 402-406, 1996; Strynadka, et al. Nature Struct. Bio. 3: 233-239, 1996) and typically require knowledge of the 3D structure of both the ligand and receptor. Interacting ligand:receptor surfaces are described by different physical properties (e.g., Connolly surfaces, grids, protein slices, property-vectors, etc.) to allow the identification of geometrically complementary regions in the ligand and receptor.

The goals of molecular docking are to reconstruct the conformation of a bound ligand:receptor complex, starting from the structures of the unbound ligand and receptor molecules, and to predict the affinity of the interaction between the ligand and receptor. Despite the complexity of this problem, several approximations lead to effective and yet computationally feasible approaches.

Conceptually, docking can be split into two related tasks. First, a variety of feasible ligand:receptor complexes are generated using a search algorithm. Second, these structures are each scored to estimate binding affinity and to evaluate the likelihood of a particular ligand:receptor structure forming. Scores for a number of alternative complex's structures can be ranked, with the highest scoring structure being predicted to represent the actual ligand/receptor complex's. Where a large number of scores are to be calculated for ranking, it is important for the scoring function to be as fast as possible while retaining accuracy.

To simplify the computational complexity of docking and reduce configuration space to a manageable level, search algorithms typically focus only on the binding site of a receptor on the premise that it is pointless to attempt to dock a ligand with a region in the receptor which is known not to be involved in binding. A number of algorithms are available to predict binding sites. SPHGEN™ is an example of an algorithm which defines the binding site of a receptor by using clusters of overlapping spheres to represent the solvent-accessible surface of the receptor (Chen, et al., Biochemistry 36: 11402-11407, 1997), creating a negative image of surface invaginations. The radii of the spheres are proportional to the concavity of the receptor surface: flat regions are represented by larger spheres while small spheres are generated in highly featured regions.

Spheres are constructed using a molecular surface program, such as the one described by Richards (Annual Review of Biophysics and Bioengineering, 6: 151-176, 1977) and Connolly (Appl. Crystallogr. 16: 548-558, 1983; Science 221: 709-713, 1983). Spheres are calculated over the entire surface of a macromolecule (e.g., receptor or ligand), producing approximately one sphere per surface point. Spheres are then selectively eliminated by filtering to keep only the largest sphere associated with each surface atom. The filtered set is then clustered on the basis of radial overlap between the spheres using a single linkage algorithm. This creates a negative image of the molecular surface (e.g., such as a receptor surface), where any invagination is characterized by a set of overlapping spheres. These sets, or “clusters,” are sorted according to numbers of constituent spheres, with the largest cluster in a receptor molecule (i.e., comprising the most spheres) typically identified as representing a ligand binding site.

After identifying potential binding sites on a receptor, a docking program is used to simulate different conformations of a receptor, and to select a conformation which can interact with a ligand using a minimum amount of energy. The docking package, DOCK™, like SPHGEN, uses a sphere definition of a target binding site to guide the positioning of ligands (Kuntz, et al.,. J. Mol. Biol. 161: 269-288, 1982). The centers of spheres are used to identify possible positions for ligand atoms in a putative binding site of a receptor. Selected ligand atoms are mapped onto subsets of sphere centers with internal distances between sphere centers approximating the distance between ligand atoms. Each mapping attempt or “docking run” orients a ligand in the site. A minimum of four atom-sphere center pairs is required to define a unique configuration for a ligand, and thus thousands of orientations are possible.

Following transformation of a ligand's coordinates (each coordinate representing the position of an atom within the ligand) into a particular orientation, the orientation is scored by evaluating the extent of shape complementarity between the receptor and the ligand using a scoring function which approximates van der Waals interactions between atoms. Ligand atoms docked within attractive distances of receptor atoms are assigned positive scores, while orientations in which ligand atoms overlap receptor atoms are assigned negative scores and discarded as unlikely to form in vivo.

In the biological field, docking has typically been used for predicting protein/protein and protein/small molecule interactions, with only a few reports of docking a ligand to RNA. Chen, et al., supra, describe the application of SPHGEN to the analysis of an RNA molecule to define its binding site for a ligand. The binding site of RNA molecule/receptor is represented by spheres which outline the portion of the RNA molecule accessible to solvent. Rigid body orientation of a ligand to overlap the “shape” defined by the spheres is used to identify complementarity between the ligand and RNA molecule. Structures were scored on the basis of molecular mechanics, using a modified version of AMBER force field with DOCK (Kuntz, et al, supra; Ewing and Kuntz, J. Comput. Chem. 18(9): 1175-1189, 1997).

Filikov, et al. (J. Comput. Aided Molec. Des. 14(6): 593-610, 2000) describe a method similar to that described in Chen, et al., supra, where DOCK (with its molecular mechanics scoring capabilities) is extended and applied to RNA. In Hermann, et al., (J. Med. Chem. 42: 1250-1261, 1999), electrostatic patches on an RNA molecule are identified using Brownian dynamics. A pharmacophore-type motif is identified and aminoglycosides are docked using the motif as a guide to place their amino groups.

In Srinivasan, et al. (Folding & Design 1: 463-472, 1996), peptides are docked to RNA using molecular mechanics energy minimisation. Leclerc and Cedegren (J. Med. Chem 41: 175-182, 1998) describe a docking method for RNA which uses a 3D pharmacophore search and molecular dynamics optimisation method. Leclerc and Karplus (Theo. Chem. Accounts 101: 131-137, 1999) describe multiple copy simultaneous search (MCSS)-based docking of arginine and aminoglycosides to the TAR RNA using a fragment-based molecular mechanics approach.

Where scoring functions have been used in RNA docking (see, e.g., Chen, et al., supra), these have been based on molecular mechanics, considering van der Waals and electrostatic interactions between a ligand and the RNA receptor (e.g., such as by using Dock 3.5, described by Filikov, et al., supra). While pseudo-energies have been used successfully to score protein docking configurations (e.g., such as by using the LUDI program, described in Böhm, J. Comp. Aid. Molec. Des. 12: 309, 1998; FlexX™, described in Rarey, et al. J. Mol. Biol. 261: 470-489, 1996; at www.tripos.com, and available from Tripos, Inc., and Hammerhead, described in Welch, et al., Chem. Biol. 3(6): 449-462, 1996), these scoring functions have proved problematic in identifying feasible ligand: RNA receptor interactions because they require training against empirically-determined ligand: receptor complexes. The relative lack of ligand:RNA structures has meant that this approach has not been seen as suitable for RNA.

### SUMMARY OF THE INVENTION

It is an object of the invention to provide improved methods for molecular docking, particularly for docking ligands to RNA receptors. In particular, the invention provides improved methods for defining active sites in receptors, improved scoring functions for scoring ligand:RNA complexes, and methods of using docking to provide automated in silico library screening for RNA-binding compounds.

In one embodiment, the invention provides a method for determining the free energy of a ligand:RNA complex, using a scoring function for calculating pseudo-energy values of the ligand:RNA complex's structure. Preferably, the scoring function calculates pseudo-energy values for one or more of molecular interactions between RNA and ligand selected from the group consisting of: hydrogen bonds, lipophilic interactions, ionic interactions, repulsive ionic interactions, aromatic/cation-pi interactions, and entropic costs of interaction.

In preferred embodiments, formal charges are assigned to receptor and/or ligand atoms prior to scoring and the pseudo-energy values are weighted. The pseudo-energy value for a hydrogen bond is a function of the donor-acceptor bond length, the acute angle around the donor hydrogen, and the acute angle around the acceptor atom. The pseudo-energy value for aromatic/cation-pi interactions is a function of the average perpendicular distance from a ring center to the other ring plane and is calculated by determining this distance for each ring center and the average slip angle between the rings. The pseudo-energy value for lipophilic interactions is a linear version of a Lennard-Jones potential function. Preferably, this function has an attractive part and a repulsive part.

In one embodiment, the invention provides a method for identifying a putative binding site cavity in a receptor comprising the following steps: placing a grid (e.g., a cube) comprising a plurality of grid points over a three-dimensional representation of the receptor; identifying as an excluded volume one or more grid points which lie within the van der Waals surface of the receptor; removing any grid points belonging to the excluded volume, centering a large sphere over all grid points and determining whether one or more grid points within the large sphere overlaps with the grid points within the excluded volume of the receptor; removing any grid points which do not overlap; centering a small sphere over all remaining grid points, determining whether one or more grid points within the small sphere overlap with the excluded volume of the receptor, and identifying any non-overlapping grid points as representing a putative binding site cavity within the receptor.

In a another embodiment, the method further comprises the step of dividing grid points identified as representing a putative binding cavity into contiguous cavity regions. In a further embodiment, the method further comprises filtering the cavities to remove cavities smaller than a selected minimum size. In still a further embodiment of the invention, the method further comprises the step of eliminating cavities having a center of mass further than a maximum distance from a designated active site center; and identifying any remaining cavities as the binding site of the receptor for the ligand. The receptor can be a protein, a nucleic acid, a carbohydrate, or a lipid. In one embodiment, where the receptor is an RNA molecule.

In one embodiment, the grid is a cube and the spacing between corners of the grid is 0.5 Å. In one embodiment, the selected minimum size is around 20 grid spacings.

In one embodiment, the maximum distance designated from the designated active site center is 10 Å.

The invention further provides a method for docking a ligand to an RNA molecule, wherein the free energy of a ligand:RNA complex's structure is calculated using a scoring function for calculating pseudo-energy values of molecular interactions between the ligand and the RNA molecule. In one embodiment, the method comprises docking a plurality of ligands to a plurality of RNA molecules. In another embodiment, the method of dockingcomprises using Monte Carlo simulated annealing. In a further embodiment, the method comprises using distance restraints are used to keep ligand atoms within a specified distance of at least one of the binding site grid points. In still a further embodiment, the specified distance is 7 Å.

The invention also provides a method of screening a library of ligand structures to identify a ligand which interacts with an RNA molecule, the method comprising: docking a structure from the library against the RNA receptor to simulate the formation of a ligand:RNA complex and calculating the free energy of the ligand:RNA complex's structure using a scoring function for calculating pseudo-energy values of one or more interactions between the ligand and the RNA molecule. In one embodiment, the scoring function is used to identify a structure having minimal energy requirements. In another embodiment, the RNA is a drug target, and the ligand is a candidate lead drug. In a further embodiment, the ligand inhibits translation of the RNA molecule in vivo.

The invention also provides a method for defining a binding site in a biological macromolecule based on a two-sphere grid, and a method for determining the free energy of a ligand/RNA structure based on pseudo-energy values. These methods can be use in docking and also in high-throughput in silico screening.

Other features and advantages of the invention are found within the description, the drawings and the following claims.

### BRIEF DESCRIPTION OF DRAWINGS

The objects and features of the invention can be better understood with reference to the following detailed description and accompanying drawings.

FIG. 1 illustrates how the average perpendicular distance from each ring center to the other ring plane (A) and the average slip angle between the rings (B) are calculated for aromatic interactions according to one embodiment of the invention.

FIG. 2 is a graph plotting calculated binding energies vs. root mean square (RMS) deviations of a recognition fragment from an experimental structure according to one embodiment, i.e., in this embodiment, for 100 docking runs of arginine into an arginine aptamer. The point to the far left of the graph represents the native structure prior to energy minimisation, hence its poor score.

FIGS. 3A and 3B are overlays of the highest scoring docked structures obtained in docking experiments using a tobramycin (tob) aptamer (FIG. 3A) and an adenosine monophosphate (AMP) aptamer (FIG. 3B).

### DESCRIPTION

The invention provides a method for defining a binding site in a biological macromolecule, such as a receptor, and, in one embodiment, utilizes a two-sphere grid to map a receptor and to locate cavities likely to be accessible to ligands. A method for determining the free energy of a ligand:RNA complex's structure based on pseudo-energy values is also provided. In one embodiment, this method is used in identifying ligands from a library which interact with an RNA receptor of interest, by identifying a ligand:RNA structure having minimal energy requirements.

### Definitions

In order to more clearly and concisely describe and point out the subject matter of the claimed invention, the following definitions are provided for specific terms that are used in the following written description and the appended claims.

As used herein, the term “docking” is used to refer to an in silico method of characterizing receptor:ligand interactions. Docking comprises orienting a ligand structure with a putative binding site structure in a receptor and evaluating the biochemical stability of the interaction (e.g., the amount of energy required to form a ligand: receptor complex). Evaluating is performed using an algorithm comprising both searching and scoring functions which searches for possible receptor conformations and scores the conformation according to its likelihood of forming based on energy considerations (i.e., lower free energy values for a conformation would have a higher likelihood of forming in vivo) and/or the degree of shape complementarity between the receptor and the ligand.

As used herein, a “receptor” is any biological macromolecule to which another molecule can bind (naturally or after engineering the molecule). Receptors according to the invention, include, but are not limited to, proteins, polypeptides, nucleic acids (DNA, RNA, and modified forms thereof), carbohydrates and lipids. A receptor has at least one binding site for a ligand.

As used herein, a “cavity” refers to a portion of a receptor which has the potential to receive and interact with a ligand. A “binding cavity” is a cavity which has a high likelihood of receiving and interacting with a native ligand, based on energy minima evaluations. The shape of a binding cavity generally corresponds to the negative shape of a ligand and comprises a binding site for the ligand.

As used herein, a “binding site” or “active site” refers to an atom or group of atoms within a receptor which form chemical contacts with a ligand (e.g., such as ionic bonds, van der Waals interactions, and the like) which stabilize a ligand: receptor interaction (e.g., making the interaction more energetically favored).

As used herein, “a ligand” is any molecule which can be bound by a receptor, and includes, but is not limited to, proteins, polypeptides, nucleic acids (DNA, RNA, and modified forms thereof), carbohydrates and lipids. A ligand has at least one site which is complementary to the binding site of a receptor (e.g., forming energetically favored interactions with the binding site).

As used herein, “native” and “non-native ligands” of a receptor refer, respectively, to ligands which are found in vivo in living cells, and ligands whose structures are based on in silico modeling predictions and are not found in vivo in living cells.

As used herein, “van der Waals' forces” refer to interatomic or intermolecular forces of attraction due to the interaction between fluctuating dipole moments associated with molecules not possessing permanent dipole moments. These dipoles result from momentary dissymmetry in the positive and negative charges of the atom or molecule, and on neighboring atoms or molecules. The dipoles tend to align in antiparallel direction and thus result in a net attractive force.

As used herein, a “van der Waals surface” is a surface which provides the most favorable intramolecular van der Waals' forces. The van der Waals surface can be represented in various ways including as a volume map, a dot surface, or a Connolly surface.

As used herein, a “van der Waals volume” refers to a volume of a molecule which is impenetrable by other molecules with thermal energies at ordinary temperatures. Calculations of van der Waals volumes are described in Bondi, J. of Physical Chemistry, 68(3): 441-451, 1964, the entirety of which is incorporated herein by reference.

As used herein, the term “sphere” refers to a graphical representation of a portion (e.g., atom or groups of atoms) of a molecule. A sphere comprises a pre-selected radius, e.g., such as the van der Waals radius of an atom (or groups of atoms, such as the atoms in a small ligand, ligand fragment, or solvent).

As used herein, “an active site sphere” or “binding site sphere” refers to a sphere whose center point is at the active site or binding site of a binding cavity.

ΔGᵣₒₜNᵣₒₜ+ΔG₀—Entropic Cost of Binding

The entropic cost of ligand binding is estimated from the number of ligand rotatable bonds (Nᵣₒₜ) plus a constant (ΔG₀=+5.4 kJmol⁻¹) that represents the loss of translational and rotational freedom of the ligand. This term is similar to that used in Böhm, et al., supra, FleXX (Rarey, et al., supra), etc.

IV. Automated in Silico Library Screening for RNA-binding Compounds

Unlike previous methods (e.g., Chen, et al., supra), the methods according to the invention are ideally suited to high-throughput in silico screening of a large number of ligands against an RNA target of interest (e.g., several thousand ligands per day). This is also true when comparing with the method of Filikov, et al., supra, which uses methods derived from DOCK and molecular mechanics.

Therefore, in one embodiment, the invention further provides a method for screening a library of ligand structures to identify ligands which interact with an RNA receptor of interest. The method comprises docking each of a plurality of structures representing ligands from the library against the RNA receptor structure and calculating the free energy of a ligand:RNA complex's structure using a scoring function for calculating pseudo-energy values of the ligand:RNA complex's structure to identify a structure having minimal energy requirements.

Ligands may be designed de novo and their structures saved in a computer readable format, such as SD or MDL formats. Alternatively, many vendors provide their catalogue of compounds in a computer readable format.

In one embodiment, a 3D structure of a ligand is generated from a 2D representation using a program such as CORINA (e.g., Gasteiger, et al., Tetrahedron Comp. Method. 3: 537-547, 1990); CONCORDE, or InsightII (www.msi.com).

Using the scoring function of the invention, a complete docking run on a low-molecular weight ligand takes 5-15 seconds (SGI MIPS R10K 250 MHz), depending on the number of ligand interaction centers.

To optimise the scoring function for high-throughput methods, the receptor's rigidity and the short-range nature of the interaction scoring functions can be exploited. It is thus useful to pre-calculate neighbour lists for a receptor. For example, an indexed grid centered.

As used herein, a “maximum distance from an active site sphere” (or grammatical equivalents of this term) refers to the maximum distance of a ligand may have from an active site and still have favorable energy interactions with the binding site.

As used herein, the term “molecular surface” refers to a surface defined by the inward surface of a hypothetical sphere (a “probe sphere”) as it rolls over a protein molecule (see, e.g., Richards, Annual Review of Biophysics and Bioengineering, 6: 151-176, 1977; Greer and B. L. Bush, Proc. Natl. Acad. Sci. USA 75: 303-307, 1978, the entireties of which are incorporated by reference herein). A molecular surface may be subdivided into a “contact surface”, and a “reentrant surface.” The “contact surface” is the part of the van der Waals surface of the atoms that the probe sphere can touch, while the “reentrant surface” is a space defined by the inward-facing part of a probe sphere when it is simultaneously touching three atoms and the volume swept out by the probe sphere as it rolls around a pair of atoms (see, e.g., Molecular Surface Package Implementation Manual, Version 3.9, Feb. 10, 2000, 1259 El Camino Real, #184,Menlo Park, Calif. 94025 U.S.A).

As defined herein, the “binding mode” refers to molecular interactions between a ligand and receptor.

As used herein, “aromatic/cation-pi interactions” refer to interactions between an aromatic ring and a positively charged molecule. An aromatic/cation-pi interaction can be either attractive or repulsive, as determined by the electrostatic potential surface of the aromatic ring itself (see, e.g., Dougherty, Science 271: 163-168, 1996, the entirety of which is incorporated by reference herein).

As used herein, “configuration space” is a space which specifies the values of all potentially accessible physical degrees of freedom of a molecule.

As defined herein, “simulated annealing” refers to a method of defining minimal energy zones in a molecule using a random walk, in which a small, random perturbation from a given, current position is selected as a move, and the energy change associated with making this move is calculated. If the energy change is less than or equal to zero, then the move is accepted. A random move having an energy change which is greater than zero however, is accepted with an acceptance probability of: p=e−^{δE/T}, where T is the “annealing temperature.” The larger the value of “δE,” the smaller the acceptance probability (see, e.g., as described in U.S. Pat. No. 5,241,470, the entirety of which is incorporated herein by reference).

As used herein, the term “docking run” refers to an in silico simulation in which the orientations of sphere(s) representing a ligand within a putative binding cavity within a receptor are modified and the energy state of a ligand:receptor complex is determined.

As defined herein, an “aptamer” is an nucleic acid which binds to a target molecule with a much higher degree of affinity than it binds to non-target materials in a sample. The Kd of an aptamer for its target molecule is at least about 50-fold greater than for non-target molecules in a sample. As used herein, the term “aptamer binding” refers to non- “Watson-Crick” binding interactions.

I. Defining a Binding Site in a Biological Macromolecule

To simplify the computational complexity of docking, it is helpful to identify the most important active site cavities in a biological macromolecule, e.g., such as a receptor, and to restrict the sampling of ligand interactions to these regions. In one embodiment, therefore, the invention provides a method for defining a binding site in a receptor which utilises a two-sphere grid method of mapping to locate cavities within the receptor likely to be accessible only to ligands.

In one embodiment, the crystal coordinates of a characterized target receptor are obtained, e.g., from a protein database such as the Research Collaboratory for Structural Bioinformatics Database, (see, www.rcsb.org/pdb), the Brookhaven Protein Data Bank (gopher://pdb.pdb.bnl.gov/110), or the Cambridge Crystallographic Database (The Cambridge Crystallographic Data Center, 12 Union Road, Cambridge CB2 1EZ, U.K.). However, in another embodiment, the coordinates of an uncharacterized receptor (e.g., such as an RNA molecule) are determined by methods routine in the art, e.g., by X-ray diffraction, or NMR, or through the use of physical models.

In one embodiment, a 3D representation of the receptor may be generated using a program such as ms which reads atomic coordinates in Protein Databank Format (e.g., Connolly, Appl. Crystallogr. 16: 548-558, 1983; Connolly, Science 221: 709-713, 1983, Connolly, J. Mol. Graphics 11: 139-141, 1993, the entireties of which are incorporated by reference herein) or using other programs, such as described in Agishtein, J. Biomolec. Struct. & Dynamics 9(4): 759-768, 1992; Colloc'h, et al., J. Mol. Graphics 8: 133-140, 1990;. Colloc'h, et al., J. Mol. Graphics 5: 170, 1988;; Connolly, J. Mol. Graphics 3: 19-24, 1985; Connolly, J. Am. Chem. Soc. 107: 1118-1124; Connolly, et al., Comput. Chem. 9: 1-6, 1985; Connolly, J. Appl. Crystallogr. 18: 499-505, 1985, Connolly, J. Mol. Graphics 4: 3-6., 1986; Connolly, J. Mol. Graphics 4: 93-96, 1986; Connolly, Visual Computer 3: 72-81, 1987, Connolly, Biopolymers 32: 1215-1236, 1992; Duncan, et al., Biopolymers 33: 219-229, 1993; Duncan, et al., Biopolymers 33: 231-238, 1993; Eisenhaber, et al., J. Comp. Chem. 14(11): 1272-1280, 1993; Eisenhaber, et al., J. Comp. Chem. 16(3): 273-284, 1995; Gibson, et al., Molec. Phys. 64: 641-644, 1988; Guerrero-Ruiz, et al., Computers Chem. 15(4): 351-352, 1991; Heiden, et al., J. Comp. Chem. 14(2): 246-250, 1993; Heiden, et al., J. Comp. Aided Mol. Design 4: 255-269; Kundrot, et al., J. Comp. Chem. 12(3): 402-409, 1991; Lin, et al., Proteins: Structure, Function, and Genetics 18: 94-101, 1994; Pascual-Ahuir, et al., J. Comp. Chem. 11(9): 1047-1060, 1990; Perrot, et al., J. Comp. Chem. 13(1): 1-11, 1992; Perrot, et al., J. Mol. Graphics 8: 141-144, 1990; Petitjean, J. Comp. Chem. 15(5): 507-523, 1994; and Petitjean, J. Comp. Chem. 16(1): 80-90, 1995, for example, the entireties of which are incorporated by reference herein.

A 3D grid (e.g., such as a cube-shaped grid) is then superimposed over the 3D representation of the receptor. In one embodiment, the dimensions of the grid are selected based on the size and dimensions of a receptor being evaluated. Typically, the grid will have a spacing between corners of between 0.1 Å and 2 Å. A preferred grid is cubic and has a spacing of between 0.2 Å and 1 Å, and most preferably a spacing of approximately 0.5 Å. The grid is preferably placed over the representation of the receptor using Cartesian coordinates. In one embodiment, a grid is centered on a putative active site region for a receptor. As the grid becomes finer (i.e., as the spacing between corners decreases), more detailed information relating to a binding site can be obtained. However, information is achieved at the expense of computation power and a balance between required detail and computation power should thus be optimized for any particular situation.

In one embodiment, the grid comprises a plurality of x-, y- , z-coordinates, each x-, y-, z-coordinate representing a point in 3D space (i.e., a “grid point”). Grid points which lie within the van der Waals volume of the receptor (e.g., calculated using standard atomic radii), and therefore unlikely to be involved in ligand binding, are identified as forming an “excluded volume” which is eliminated from further analysis.

The remaining grid points (i.e., identifying portions of the receptor possibly involved in binding to a ligand) are checked for accessibility to a large sphere having a selected radius. In one embodiment, the radius of the large sphere is in the range 3-5 Å, and preferably about 4 Å. Any grid points in the large sphere which do not overlap with grid points remaining after removing excluded volume grid points are themselves removed from further analysis. The step of determining accessibility to a large sphere has the effect of eliminating any large cavities and of smoothing convex regions of a receptor surface.

Any grid points still remaining (representing site(s) of putative ligand:receptor interactions) are checked for accessibility to a smaller sphere having a pre-selected radius (e.g., a sphere representing a small ligand, ligand fragment, or solvent molecule, such as H₂O). In one embodiment the radius of the small sphere is in the range of 0.5-3 Å, preferably 1-2 Å, and most preferably around 1.75 Å.

If a small sphere centered on a remaining grid point does not overlap with receptor atoms, the grid point is identified as belonging to a cavity. An ensemble of adjacent remaining grid points defines the boundaries of a binding cavity in the receptor, i.e., a site of putative ligand binding (e.g., a binding site). Depending on the complexity of the receptor surface, there may be more than one binding cavity and binding site.

In combination with the step of determining accessibility of the receptor to a large sphere, the subsequent step of identifying accessibility of the receptor to a small sphere, identifies coordinates of the grid/regions of the receptor molecule corresponding to “deep” cavities, i.e., those that are accessible to small spheres but not larger ones.

The method for defining a binding site according to the invention may optionally comprise either, or both, of the following additional two steps: (1) filtering cavities to remove those with a smaller than minimum size; (2) defining the coordinates of an active site center and removing cavities with a center of mass further than a maximum distance from the designated active site center. The minimum size used in optional step (1) is an empirical estimate (based on the nature of the receptor and ligand) and, in one embodiment, is in the range of 5-50 grid points, preferably 10-30 grid points, and most preferably around 20 grid points. In one embodiment, given a 0.5 Å grid, the minimum cavity size will be 10 Å.

If information is available on the location of the active site or binding site, 3D coordinates of the active site center can be identified relative to the coordinates of the atoms of the whole receptor. A maximum distance can then be used to define the extent of the active site in optional step (2). The center point of the active site and the maximum distance together define an “active site sphere.” The assumption is that regions of a binding cavity which are remote from the center of the binding site (more than maximum distance) are predicted not to be critically involved in binding. The maximum distance will typically be in the range 1-50 Å, preferably 5-20 Å, and most preferably around 10 Å. The center point can be defined by using the center of mass of the receptor, or prior information relating to the location of the active site.

Unlike SPHGEN, the method according to the invention avoids the need for manual intervention (e.g., input of empirically determined parameters by a user) in order to guide the algorithm in identifying a binding site(s), although manual guidance can be used if desired. Cavity mapping is therefore improved. In comparison with the binding site calculation method described in Afshar, et al., J. Mol. Biol. 244: 554-571, 1994, the method according to the invention uses grid points and no surface calculation is performed, resulting in improved computation speeds. Further, in comparison with the method implemented in Cerius² (described at www.msi.com), the use of two spheres instead of a single sphere as in Cerius², allows the filtering of ripples at the bottom of cavities, thus simplifying the surface features of a cavity and increasing the efficiency of docking.

II. Docking Methods

After defining the binding site of a receptor for a ligand, a ligand structure (i.e., a representation of a ligand) is docked to the binding site, such as by using any of the standard docking algorithms available. In one embodiment, after a binding site is identified, the search function of a docking program is employed to sample the degrees of freedom of putative ligand:receptor complexes formed in sillico. In some embodiments of the invention, the ligand is considered to be rigid, and only six degrees of freedom need to be sampled. However, in other embodiments, alternate conformations of the ligands (obtained by varying each torsion angle that can be varied) are considered, adding complexity (e.g., computational time) to the search.

In one embodiment, the search algorithm may use molecular mechanics, such as energy minimisation (e.g., Sezerman, et al., Protein Science 2: 1827-1843, 1993), molecular dynamics (e.g., Luty, et al., J. Comput. Chem. 18: 723-743, 1995), or multiple copy simultaneous search (e.g., Miranker et al., Proteins 11: 29-34, 1991). Monte Carlo simulated annealing programs can also be used (Caflish, et al. J. Comput. Chem 18: 723-743, 1996; Goodsell, D. S. and Olson, A. J., Proteins: Struct. Funct. Genet., 1990, 8, 195-202, describing the AUTODOCK program, available from Scripps Research Institute, La Jolla, Calif.), as well as combinatorial search programs (e.g. in-site combinatorial search programs, or rotamer search programs, as described in, for example, U.S. Pat. No. 5,854,992), or genetic algorithms.

In other embodiments, ligands are assembled from fragments, such as by using the FlexX program (Rarery, et al., supra), Dock 4.0 (Kuntz, et al, supra; Ewing and Kuntz, supra) the Ludi program (Böhm, et al., supra, available from Biosym Technologies, San Diego, Calif.), the Hammerhead program (Welch, et al., supra), Growmol (Bohacek and McMartin, SIAM J. Math. Anal. 116: 147-179, 1995), or the Hook program (Eisen, et al., Proteins: Struct. Funct. Genet. 19: 199-221, 1994). (The entireties of all the above references are incorporated herein.)

Molecular mechanics algorithms are preferred, because they can be readily used to sample the conformational space of the receptor and optimise the geometry of docked ligands. Furthermore, their theoretical basis allows the interpretation of the physico-chemical features that are relevant to a particular prediction. Monte Carlo algorithms are particularly preferred.

The method for defining putative binding sites of the receptor molecule according to the invention results in sets of grid points representing the binding sites. In one embodiment, distance restraints are used to keep ligand atoms within a specified distance of at least one of these grid points. Atoms further away from a binding site thus do not contribute to the scoring function. However, in some embodiments, it may be preferred to retain calculation of the repulsive van der Waals' term for these atoms. In one embodiment, the specified distance is in the range of 1-20 Å, preferably 5-10 Å, and more preferably around 7 Å. This provides a non-spherical docking region that is closely moulded to the shape of the binding site cavity.

In one embodiment, a ligand is placed or oriented within one of the receptor's putative binding site cavities at the start of each independent docking run. If multiple cavities are present, a cavity is selected at random, preferably with a probability proportional to its size (e.g., a size that approximates the size of the ligand would be given a high probability) and a ligand center of mass is placed either at the cavity center of mass or on a randomly selected cavity grid point. The principal axes of the ligand are then aligned either with the principal axes of the cavity or along a randomly selected axis. The internal conformation of the ligand is preferably retained from the best scoring bound conformation obtained from previous run(s).

In one embodiment, a preferred probability for placing the ligand in any given cavity is identified by determining the ratio between the cavity volume and the total volume of all cavities. In another embodiment, it is preferred to align the ligand center of mass with the cavity center of mass, while in still another embodiment, it is preferred to align the ligand principal axes with the cavity principal axes.

In one embodiment, once the ligand is placed or docked, it is subjected to Monte Carlo sampling. A typical Monte Carlo trial comprises the steps of: translating the ligand along a random vector selected from a spherical distribution; rotating the ligand around a random axis vector (also selected from a spherical distribution) through the ligand center of mass; and torsionally rotating one of the ligand's rotatable bonds. In one embodiment, the maximum step sizes for each of the three steps (i.e., translating the ligand, rotating the ligand around the random axis, and torsionally rotating the ligand's rotatable bonds) will be chosen to give a Metropolis acceptance rate of approximately 0.5 (e.g., Metropolis, et al., J. Chem. Phys. 21, 1087,1953).

In one embodiment, Monte Carlo sampling is preferably used within a non-adaptive simulated annealing cooling schedule for locating low energy bound conformations. A typical schedule is divided into a number of phases. In each phase, a fixed number of Monte Carlo trials are performed in a series of simulated constant temperature blocks. At the end of each temperature block, the sampling temperature is reduced by a constant factor (geometric cooling). In addition, the maximum Monte Carlo step sizes may be reduced (e.g., halved) if the acceptance rate falls below a threshold (e.g., 0.25) over a particular block. The next phase may begin from the lowest scoring ligand conformation found during the previous phase.

Standard Monte Carlo protocols and parameters can be found in Morris, et al., J. Comput. Aided Mol. Des. 10: 293-304, 1996; Liu, et al., J. Comput. Aided Mol. Des. 13: 435-451, 1999; and Cummings, et al., Protein Science 4: 885-899, 1995, the entireties of which are incorporated herein.

III. Scoring Functions for Ligand:RNA Complexes

To estimate the likelihood that a ligand:receptor complex's structure identified in silico represents a structure actually formed in vivo, structures identified in docking runs can be scored, or ranked, to identify the structure most likely to form in vivo. In one embodiment, the invention provides a method for predicting the free energy of a ligand:RNA complex's structure (“pseudo-energy”) as a means of scoring. This method utilises a simplified empirical scoring function for calculating energy values of the ligand:RNA complex's structure. The need for molecular mechanics calculations is thus avoided and computational time is hugely reduced. In another embodiment, pseudo-energy value scoring is used during the docking procedure itself to calculate the free energy of a ligand:RNA structure formed. By using this scoring method, native and non-native ligands can be distinguished (e.g., non-native ligands will form high free energy ligand:RNA structures, while native ligands will form low free energy ligand:RNA structures).

Preferably, the scoring function calculates pseudo-energy values for one or more (and preferably all) of the following interactions between RNA and ligand: hydrogen bonds, attractive lipophilic interactions, repulsive lipophilic interactions, attractive ionic interactions, repulsive ionic interactions, aromatic/cation-pi interactions, and entropic costs of interaction. These terms may contribute to the scoring function equally, but are preferably weighted.

Preferably, the method of the invention uses the scoring function set out as equation (I):

$$Δ {G}_{binding} = \{ \begin{matrix}Δ {G}_{H - bond} {\sum }_{H - bonds} {f}_{1} ( Δ R ) \cdot {f}_{1} ( {Δα}_{Don} ) \cdot {f}_{1} ( Δ {α}_{Acc} ) \cdot {f}_{2} ( {c}_{Don} , {c}_{Acc} ) \cdot {f}_{3} ( {N}_{Neighb} ) + \\ Δ {G}_{ionic} {\sum }_{ionic} {f}_{1} ( Δ {R}_{ij} ) \cdot {f}_{2} ( {c}_{i} , {c}_{j} ) \cdot {f}_{3} ( {N}_{Neighb} ) + \\ Δ {G}_{arom} {\sum }_{arom} {f}_{1} ( Δ \overline{{R}_{perp}} ) \cdot {f}_{1} ( Δ \overline{{α}_{Slip}} ) + \\ Δ {G}_{lipo} {\sum }_{lipo} {f}_{1}^{′} ( Δ {R}_{ij} ) + \\ Δ {G}_{rot} {N}_{rot} + \\ Δ {G}_{0}\end{matrix} \qquad {equ}^{n} ( I )$$

Equation (I) is a scoring function for the binding free energy of a ligand:RNA complex's as a weighted sum of defined interactions (c.f., Böhm, et al. supra). The interactions which are included in the equation, the functional forms used to describe each interaction, and the free energy weightings assigned to each interaction were determined empirically by fitting the parameters of the equation to known affinities for ligand:RNA complexes of known structure. Due to the relative scarcity of high-resolution RNA-ligand structures, however, a full statistical approach using a large training set is not practical. Therefore, in one embodiment, the scoring function is optimised against RNA aptamers (RNA molecules selected for their ability to bind with a pre-selected target) complexed with a target ligand, with emphasis on reproducing the experimental binding modes of each ligand (e.g., using Monte Carlo docking), as well as predicting the binding affinity of a ligand for an individual RNA aptamer/receptor. The ability of the scoring function of the invention to discriminate between native and non-native ligands has also been demonstrated through the calculation of a full cross-docking matrix of binding affinities, in which each ligand is docked into each aptamer in the training set (see Examples).

Typical weightings and parameters for the free energy terms in the scoring function are:

| Term | ΔG (kJmol⁻¹) | X | X₀ | ΔXₘᵢₙ | ΔXₘₐₓ |
|---|---|---|---|---|---|
| H-bond | −3.4 | R | 1.9 Å | 0.25 Å | 0.6 Å |
| | | α_{Don} | 180° | 30° | 80° |
| | | α_{Acc} | 150° | 30° | 70° |
| Ionic | −1.0 | Rᵢⱼ | (ΣR_{vdW}) + 0.6 Å | 0.25 Å | 0.6 Å |
| Arom | −1.6 | Rₚₑᵣₚ | 3.5 Å | 0.25 Å | 0.6 Å |
| | | α_{Slip} | 0° | 20° | 30° |
| Lipo | −0.12 | Rᵢⱼ | (ΣR_{vdW}) + 0.6 Å | 0.25 Å | 0.6 Å |
| Rot | +1.0 | | | | |
| 0 | +5.4 | | | | |

where X, X₀, ΔXₘᵢₙ, ΔXₘₐₓ refer to equⁿ (II).

Formal Charges

Before applying equation (I) to a ligand/RNA structure, formal charges may be assigned to certain receptor and ligand atoms in order to modulate the strength of hydrogen bond and ionic interactions in the scoring function. Applying formal charges to ligand and receptor atoms in this way has not been used in scoring protein:ligand interactions and has not previously been done. Thus, the invention further encompasses a method of selecting atom(s) in a receptor and/or ligand and determining their associated charges.

According to the invention, atoms in the RNA receptor are assigned formal charges as follows:

| RNA Residue | Atom(s)* | Formal Charge |
|---|---|---|
| A, C, G, U | O1P, O2P | −0.5 |
| A | N7 | −0.5 |
| C | O2 | −0.5 |
| G | N7, O6 | −0.5 |
| U | O2, O4 | −0.5 |

*standard RNA atom nomenclature is used

Anionic phosphate groups and base atoms carrying a high partial charge in molecular mechanics force fields are assigned a negative formal charge, e.g., the O1P, O2P and N7 atoms in each “A” residue in the RNA receptor are assigned a formal charge of −0.5, etc. No positive charges are assigned on the receptor.

Atoms in the ligand are assigned formal charges as follows:

| Ligand Group | Atom(s) | Formal Charge |
|---|---|---|
| Primary amine | 3 H | +0.33 (⅓) |
| Secondary amine | 2 H | +0.5 |
| Tertiary amine | 1 H | +1.0 |
| Guanidinium | 5 H + C | +0.167 (⅙) |
| Imidazole | 2 H + C | +0.33 (⅓) |
| Carboxylate | 2 O | −0.5 |
| Phosphate | 2 O | −0.5 |

Formal positive charge is distributed evenly between all the polar hydrogens in any given group, except in guanidinium and imidazole groups where the central nitrogen-bonded carbon also receives an equal portion of the charge. Formal negative charge is distributed evenly between all the terminal oxygen atoms in a group.

When applying formal charges, positive ionisable groups which are usually protonated at physiological pH (e.g., amines, guanidines, imidazoles, and the like) should be protonated and negatively ionisable groups (e.g., carboxylates, phosphates, and the like) should be deprotonated.

ΔG_{H-bond}—Hydrogen Bond Interactions

Hydrogen bond geometries are defined by four atoms: donor parent—donor hydrogen·· acceptor—acceptor parent, e.g. N—H···O═C. The interaction score for a hydrogen bond is a function of the donor-acceptor bond length, the acute angle around the donor hydrogen, and the acute angle around the acceptor atom. In cases where the acceptor is bonded to more than one parent, the acceptor angle is defined to mean coordinates of all acceptor parents. This is similar to Böhm's original scoring function (Böhm, et al., supra) and FleXX's scoring functions (Rarey, et al., supra) but with the addition of an acceptor angular term, which helps to exclude unfeasible geometries in docking simulations. This term is not used in the prior art protein modeling methods because they typically deal with “real” structures which defacto do not include unfeasible geometries.

Hydrogen bond scores are scaled by function f₁ (Böhm, et al., supra), a generic geometric scoring function:

X=any geometric variable (distance, angle, plane angle)

X₀=ideal value

ΔX=|X−X₀|

ΔX_{Mim}=tolerance on ideal value

ΔX_{Max}=deviation at which score is reduced to zero

$$Δ {X}_{Max} = deviation at which score is reduced to zero \\ {f}_{1} ( Δ X ) = \{ \begin{matrix}1 & Δ X \leq Δ {X}_{Min} \\ 1 - \frac{Δ X - Δ {X}_{Min}}{Δ {X}_{Max} - Δ {X}_{Min}} & Δ {X}_{Min} < Δ X \leq Δ {X}_{Max} \\ 0 & Δ X > Δ {X}_{Max}\end{matrix} \qquad {equ}^{n} ( II )$$

Hydrogen bond scores are also scaled by function f₂ (Jain, J. Comput. Aided Mol. Design 10: 427-440, 1996, the entirety of which is incorporated herein by reference) which relies on the formal charges applied as described above:

f₂(cᵢ,cⱼ)=−sgn(cᵢcⱼ)×(1+n|cᵢ|)(1+n|cⱼ|) wherein

c₁, cⱼ=formal charges on atoms i and j

n=scale factor (0.5)

$${c}_{i} , {c}_{j} = formal charges on atoms i and j \\ n = scale factor ( 0.5 ) \\ - sgn ( {c}_{i } {c}_{j} ) = \{ \begin{matrix}+ 1 for charges of opposite sign \\ - 1 for charges of like sign\end{matrix} \qquad {equ}^{n} ( III )$$

This provides a convenient way to increase the affinity of hydrogen bonds involving formally charged donors and/or acceptors. Function f₂ ranges from 1.0 for neutral H-bonds through to 1.875 for e.g., for tertiary amine-phosphate interactions.

Hydrogen bond scores are also scaled by local receptor density, f₃ (Böhm, et al., supra):

$${f}_{3} ( {N}_{Neighb} ) = {( \frac{{N}_{Neighb}}{{N}_{Neighb , 0}} )}^{α} wherein \qquad {equ}^{n} ( IV )$$

N_{Neighb}=no. of non-H receptor atoms within 5A radius sphere of given receptor atom

N_{Neighb,0}=normalisation factor (average local receptor density)=25

α=0.5

As described in Böhm, et al., supra, function f₃ is used to distinguish between convex (exposed) and concave (cavity) regions of the receptor surface, and increases the weighting given to interactions in tightly binding cavities. Function f₃ typically ranges from 0.5 to 1.3. Together with the free energy weighting of −3.4 kJ/mol, functions f₂ and f₃ combine to give a wide range of ideal H-bond affinities, from 1.7 kJ/mol for exposed neutral interactions to 8.3 kJ/mol for highly charged interactions in tight concave cavities.

ΔG_{ionic}—Ionic Interactions

This is a specialised term, which primarily helps the correct placement of charged, planar groups such as guanidinium and imidazole. Conventional charged interactions such as amine-phosphate contacts are already accounted for in the hydrogen bonding term. The ionic term is also used to prevent close contact between polar atoms of like charge (e.g., donor-donor, acceptor-acceptor).

Distance-dependent interaction scores are calculated between the following sets of atoms:

| RNA | Ligand | Type |
|---|---|---|
| All formally charged | All formally charged non- | Attractive or |
| non-hydrogen atoms | hydrogen atoms | repulsive |
| H-bond donor hydrogens | H-bond donor hydrogens | Repulsive |
| (neutral and charged) | (neutral and charged) | |

The ideal contact (or repulsive) distance is equal to the sum of the van der Waals radii of two atoms, plus an increment. The increment is preferably close or equal to 0.6 Å. As with hydrogen bonds, the score is further scaled by the formal charges (function f₂, which changes sign for atoms of like charge) and the local receptor density (function f₃).

The invention avoids the inclusion of repulsive interactions between neutral hydrogen bond acceptors. Including these interactions causes problems with pi-pi stacking, as ligands with acceptor atoms in an aromatic ring (e.g. furan) are prevented from stacking correctly on RNA bases. Thus the invention only considers formally charged acceptors in the repulsive term.

The weighting for ΔG_{ionic} is typically relatively low (e.g. −1.0 kJ/mol) and can be considered as accounting for solvation-like interactions above and below the plane of a charged ligand group that are not accounted for by the hydrogen bonding or aromatic terms. A good example of this type of interaction is presented by arginine when bound to its aptamer.

The use of a formal charge at the center of planar groups (e.g., such as guanidinium and imidazole and their derivatives) has not previously been used in pseudo-energy scoring methods. Specifically, it is not used by Böhm et al., supra, in FlexX (Rarey, et al., supra) or in Hammerhead (Welch, et al., supra).

ΔGₐᵣₒₘ—Aromatic Interactions

The aromatic term favours parallel pi-pi stacking between aromatic rings. Guanidinium-like groups on the ligand are also included (cation-pi interactions) (e.g., as described in Dougherty, Science 271: 163-168, 1996; Ma, et al., Chem. Rev. 97: 1303-1324, 1997; Scrutton, N. S. & Raine, Biochem. J. 319: 18, 1996, Sussman, et al., Science 253: 872-879; Hendlich, M. Acta Crystallogr. D 54: 1178-1182, 1998; Wouters, Protein Sci. 7: 2472-2475, 1998;. Jorgensen, et al., J. Am. Chem. Soc. 110: 1657-1666, 1988; Jorgensen, et al. J. Am. Chem. Soc. 118: 11225-11236, 1996, the entireties of which are incorporated by reference herein).

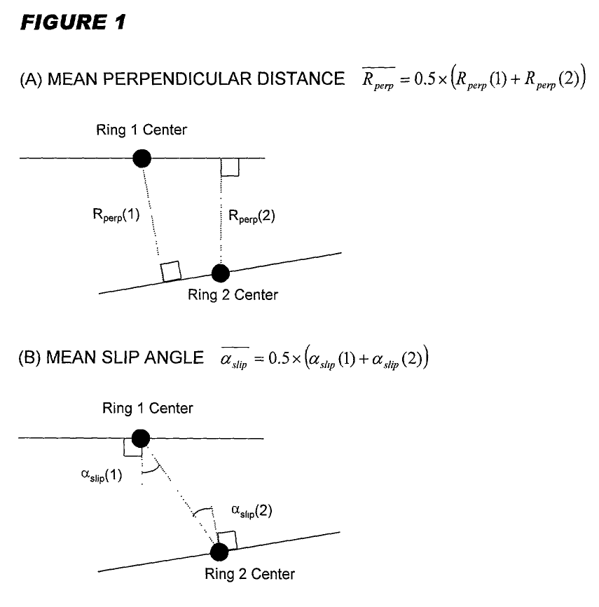

As shown in FIG. 1, the geometry is described by the average perpendicular distance from each ring center to another ring plane (ΔRₚₑᵣₚ), and by the average slip angle between rings (Δαₛₗᵢₚ). Each ring in a fused ring system is scored independently. Perpendicular stacking geometries are not accounted for.

Unlike Böhm (Böhm, et al., supra), FlexX (Rarey, et al., supra), and other pseudo-energy scoring methods, the scoring method according to the invention treats planar groups such as guanidinium and analogues as aromatic-like. This treatment is critical for the correct placement and scoring of guanidinium-like groups stacking on nucleotides, such as, for example, observed in the interaction of arginine in the TAR RNA pocket.

ΔGₗᵢₚₒ—Lipophilic Interactions

The functional form of the lipophilic interaction (f₁′) is a crude linear version of a Lennard-Jones potential (Rarey, et al., supra). The functional form of the term is similar to that used in FleXX (Rarey, et al., supra):

Rᵢⱼ=interatomic distance

R₀=ideal value

ΔR=Rᵢⱼ−R₀

ΔR_{Min}=tolerance on ideal value

ΔR_{Max}=deviation at which score is reduced to zero

Slope=10.0

$$\begin{matrix}{n}_{i} , {n}_{j} = \{ \begin{matrix}2 & methylene ( {CH}_{2} ) and methyl ( {CH}_{3} ) carbons with implicit hydrogens \\ 1 & all other atoms\end{matrix} & \\ {f}_{1}^{′} ( Δ R ) = {n}_{i} {n}_{j} * \{ \begin{matrix}Slope * \frac{Δ R + Δ {R}_{Max}}{Δ {R}_{Max} - Δ {R}_{Min}} & Δ R \leq - Δ {R}_{Max} \\ 1 + \frac{Δ R + Δ {R}_{Min}}{Δ {R}_{Max} - Δ {R}_{Min}} & - Δ {R}_{Max} < Δ R \leq - Δ {R}_{Min} \\ 1 & - Δ {R}_{Min} < Δ R \leq Δ {R}_{Min} \\ 1 - \frac{Δ R - Δ {R}_{Min}}{Δ {R}_{Max} - Δ {R}_{Min}} & Δ {R}_{Min} < Δ R \leq Δ {R}_{Max} \\ 0 & Δ R > Δ {R}_{Max}\end{matrix} & {equ}^{n} ( V )\end{matrix}$$

The function has an attractive part (equivalent to f₁), and also a repulsive part to prevent atomic overlap during docking. The slope of the repulsive potential is between 1 and 20 times the slope of the attractive potential, preferably between 5 and 15 times, and more preferably about 10 times the slope of the attractive potential. In order to promote close contact between the ligand and receptor surfaces, the lipophilic score is calculated for all receptor-ligand atom pairs (polar and non-polar).

The repulsive part of the lipophilic potential is the only score which is calculated for the ligand, and prevents unreasonable atomic clashes during Monte Carlo sampling of the ligand rotatable bonds. While a more complete calculation of ligand intramolecular strain energy can be used according to the invention (e.g., using the majority of the interaction terms outlined above), this does not give any significant improvement in the quality of docked conformations or predicted binding affinities. In the context of docking a semi-flexible ligand into a rigid receptor, it is preferred to treat all ligand conformations as equally probable rather than to attempt to rank putative binding modes by intramolecular and intermolecular free energy simultaneously.

ΔGᵣₒₜNᵣₒₜ+ΔG₀—Entropic Cost of Binding

The entropic cost of ligand binding is estimated from the number of ligand rotatable bonds (Nᵣₒₜ) plus a constant (ΔG₀+5.4 kJmol⁻¹) that represents the loss of translational and rotational freedom of the ligand. This term is similar to that used in Böhm, et al., supra, FleXX (Rarey, et al., supra), etc.

IV. Automated in Silico Library Screening for RNA-binding Compounds

Unlike previous methods (e.g., Chen, et al., supra), the methods according to the invention are ideally suited to high-throughput in silico screening of a large number of ligands against an RNA target of interest (e.g., several thousand ligands per day). This is also true when comparing with the method of Filikov, et al., supra, which uses methods derived from DOCK and molecular mechanics.

Therefore, in one embodiment, the invention further provides a method for screening a library of ligand structures to identify ligands which interact with an RNA receptor of interest. The method comprises docking each of a plurality of structures representing ligands from the library against the RNA receptor structure and calculating the free energy of a ligand:RNA complex's structure using a scoring function for calculating pseudo-energy values of the ligand:RNA complex's structure to identify a structure having minimal energy requirements.

Ligands may be designed de novo and their structures saved in a computer readable format, such as SD or MDL formats. Alternatively, many vendors provide their catalogue of compounds in a computer readable format.

In one embodiment, a 3D structure of a ligand is generated from a 2D representation using a program such as CORINA (e.g., Gasteiger, et al., Tetrahedron Comp. Method. 3: 537-547, 1990); CONCORDE, or InsightII (www.msi.com).

Using the scoring function of the invention, a complete docking run on a low-molecular weight ligand takes 5-15 seconds (SGI MIPS R10K 250 MHz), depending on the number of ligand interaction centers.

To optimise the scoring function for high-throughput methods, the receptor's rigidity and the short-range nature of the interaction scoring functions can be exploited. It is thus useful to pre-calculate neighbour lists for a receptor. For example, an indexed grid centered over an active site stores lists of all receptor atoms within a given radius of each grid point. The radius is chosen to just exceed the maximum range of any of the scoring functions (e.g., such as a van der Waals radius, +4.0 Å). In one embodiment, a lookup table is therefore provided for computing lipophilic scores.

In another embodiment, once a ligand has been identified which interacts with a receptor, the ligand is provided (e.g., synthesised, purified or purchased) and the interaction is verified experimentally, using any means known in the art to evaluate ligand:receptor interactions, including, but not limited to FRAC, NMR, filter-binding assays, gel-retardation assays, displacement assays, Surface Plasmon Resonance (SPR), and the like. In one embodiment, the invention provides a ligand identified using the library.

Ligands provided in the library can comprise proteins, polypeptides, carbohydrates, lipids, nucleic acids (e.g., DNA, RNA, and modified forms thereof), as well as drugs (e.g., chemical agents, antibiotics, and the like). In one embodiment, the X-ray coordinates of a drug target (e.g., a protein, such as the 50S or 30S subunits of ribosomes), are provided and a grid superimposed on a representation of the coordinates and/or a molecular surface. A binding site(s) is/are identified using the two-sphere method described above, and docking configurations of lead drugs/ligands (e.g., such as antibiotics, where the receptor is a ribosome) in known or novel binding cavities are determined, to identify drugs/ligands which have a predicted binding energy lower than a threshold, for example less than −20 kJ. The method provides a way to identify candidate lead drugs which are likely to bind strongly in vivo, and therefore, which are more likely to achieve a therapeutic effect. In one embodiment, where the receptor is an RNA molecule, the library is used to identify a molecule which can inhibit the translation of the RNA molecule.

### EXAMPLES

Docking Experiments

In order to demonstrate that the invention can correctly predict the binding mode of a set of ligands interacting with their receptor, docking experiments were carried out for models of five published RNA/ligand complexes (the receptors were aptamers and the ligands were arginine (Arg), citruline, adenosine monophasphate (AMP), tobramycin and flavin mononucleotide (FMN)) (Yang, et al., Nature 382(6587): 183-186, 1996; Jiang, et al., Nat. Struct. Biol. Nat Struct Biol. 5(9):769-774, 1998; Fan, et al., J. Mol. Biol. 258(3): 480-500, 1996. 3D models for RNA receptors and ligands were taken directly from Protein Database (PDB) files, with no further energy minimisation.

Cross-docking experiments were also performed for the same complexes in which binding affinities for all pairwise ligand and RNA combinations were predicted. The hypothesis was that each aptamer would have a distinct preference for its own native ligand over non-native ligands, and that each ligand will prefer to bind to its own aptamer. The cross-docking experiments were performed to test the discrimination power of the method.

100 independent docking runs were performed for each native aptamer-ligand complex, using a low-temperature cooling schedule as shown below. 50 docking runs per ligand were used in the cross-docking experiment.

| | | | No. of | | | |
|---|---|---|---|---|---|---|
| | | | Constant | | No. of | |
| | Initial | Final | Temper- | | MC | Initial Max. Step |
| | Temp | Temp | ature | Cooling | Trials/ | Sizes (Translation, |
| Phase | (°K) | (°K) | Blocks | Factor | Block | Rotation, Torsion) |
| 1 | 300 | 50 | 25 | 0.928 | 100 | 0.1 A, 10°,10° |
| 2 | 50 | 10 | 25 | 0.935 | 100 | 0.1 A, 10°,10° |

Binding Site Mapping

Binding site cavities for the RNA receptors were mapped using large and small sphere radii of 4.0 Å and 1.75 Å, respectively, and a grid resolution of 0.5 Å. Cavities with a volume less than 20 grid points, or whose center of mass was greater than 8 Å from the binding site center, were removed. In all cases, only one cavity remained. A distance restraint function was applied to keep all ligand atoms within 6 Å of any cavity grid point during docking. The results of the binding site mapping are shown below:

| | Designated Binding | Cavity | Cavity Center |
|---|---|---|---|
| | Site Center | Volume | Of Mass |
| | (mean coordinates | (no. of | Distance From |
| | of all atoms | grid | Designated Binding |
| Receptor | listed) | points) | Site Center (Å) |
| Arginine | G8:O6; G9:O2; G15:O6, N7; | 164 | 4.5 |
| | G16:O6, N7; G2O:O6, N7 | | |
| AMP | A5:N1, N3, C4, C5, C6, | 363 | 3.6 |
| | C7; | | |
| | G6:N1, C2, N3, C4, C5, C6 | | |
| FMN | G9:N3; G10:C2; U12:C4; | 150 | 2.7 |
| | G27:C6 | | |
| Tobra- | U7:O4; G12:C2 | 205 | 2.1 |
| mycin | | | |
| Citruline | U9:O2; U16:O4; A18:O4’ | 26 | 1.7 |

Native Dockings—Free Energies

Free energies of docked RNA/ligand complexes were calculated using equations I, II, III, IV and V, as described above. The lowest energy obtained in 100 dockings was taken as the result.

The structures of obtained in docking runs and the PDB file were compared to give (a) RMS deviation for the tightly bound portion of the ligand only (i.e., the recognition fragment or the fragment required for ligand binding to the receptor) and (b) RMS deviation for all ligand non-hydrogen atoms. The recognition fragment is typically much better defined in the NMR structure than the rest of the ligand.

The same equations were used to predict binding energies for the complex's as described in the PDB files, without any docking.

Both results were compared with experimental ΔG values.

TABLE 1
Docking Results For RNA Aptamers And Ligands

| | | | Calc^{d} | Calc^{d} | | RMSD |
|---|---|---|---|---|---|---|
| | | Exp^{tl} | PDB | Docked | | Recognition |
| | | ΔG_{bind} | ΔG_{bind} | ΔG_{bind} | Recognition | (All) |
| PDB | Ligand | (kJmol⁻¹) | (kJmol⁻¹) | (kJmol⁻¹) | Fragment | (Å) |
| 1koc | Arginine | −28.5 | −14.0 | −31.4 | Guanidine | 0.59 (3.73) |
| 1am0 | AMP | −28.5 | −24.0 | −32.2 | Adenine | 0.36 (2.13) |
| 1fmn | Fmn | −30.1 | −26.3 | −37.4 | Fused rings | 0.33 (0.75) |
| 2tob | Tobramycin | −45.2 | −44.8 | −38.9 | Rings 1, 2 | 0.20 (1.23) |
| 1kod | Citruline | −28.5 | −6.3 | −21.0 | Urea | 2.58 (4.20) |

Exp^{tl} = experimental;
Calc^{d} = calculated

The calculated energies in Table 1 are reported without the contribution from the repulsive lipophilic potential. The rationale for this is that any residual atomic clashes in the final conformation are likely to be an artefact of the rigid receptor/semi-flexible ligand model, and can be easily resolved in reality by a slight relaxation of the receptor or ligand (see Jain, et al., supra). Table 2 shows the breakdown of component interaction scores for each aptamer. There are significant repulsive lipophilic interactions in the PDB structures of tobramycin aptamer (2tob) in particular, and to a lesser extent in the citruline aptamer (1kod). These could be due to a combination of the particular set of van der Waals radii used and inaccuracies in the experimental structure.

TABLE 2
Breakdown Of Component Interaction Scores For Aptamer Native
Dockings

| PDB | Structure | Aromatic | H-bond | Ionic | Lipo Attr. | Lipo Rep. |
|---|---|---|---|---|---|---|
| 1koc | PDB | +0.0 | −6.8 | −5.8 | −12.7 | +4.8 |
| | Docked | +0.0 | −24.5 | −5.9 | −12.3 | +1.8 |
| 1am0 | PDB | −5.4 | −13.5 | +2.5 | −20.0 | +4.9 |
| | Docked | −4.6 | −18.1 | +0.3 | −22.2 | +0.5 |
| 1fmn | PDB | −3.0 | −12.8 | +0.0 | −26.9 | +0.0 |
| | Docked | −2.4 | −23.7 | +0.6 | −28.3 | +1.5 |
| 2tob | PDB | +0.0 | −36.9 | +0.0 | −29.3 | +17.8 |
| | Docked | +0.0 | −34.6 | +1.0 | −26.8 | +10.1 |
| 1kod | PDB | +0.0 | −7.1 | +1.2 | −11.7 | +9.8 |
| | Docked | +0.0 | −24.3 | +1.3 | −9.4 | +1.3 |

Lipo Attr. = liphophilic attraction;
Lipo Rep. = lipophilic repulsion

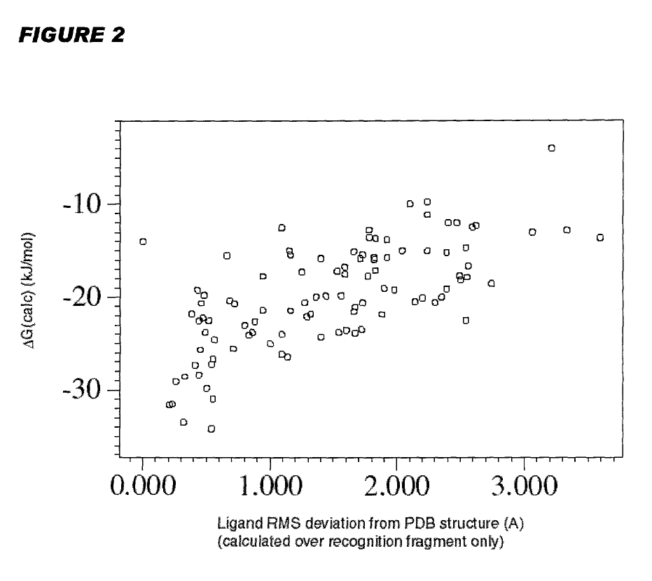

As can be seen from Table 2, binding modes and free energies were well predicted for four out of the five RNA aptamers (Arg, amp, fmn, and tobramycin). In the lowest energy docked conformation, the RMS deviation of the recognition fragment to the experimental binding mode is under 0.6 Å in all four cases. The all-atom ligand RMS deviation can be much higher, reflecting the mobility or low resolution of peripheral ligand functionality in the bound conformation. Plots of binding energy against RMS deviation of the recognition fragment for all 100 docking runs show that there are no grossly mis-docked structures with comparable energies to the experimental structure (see FIG. 2 for an example). The rank order of binding energies is reproduced from arginine (μMol) through to tobramycin (nM) aptamer.

In the case of citruline aptamer, the lowest energy ligand conformations are misdocked and the binding free energies is underestimated.

### Example 1

### Arg Aptamer

The arginine (Arg) binding cavity displayed by the Arg aptamer brings together an array of negatively ionisable N7 atoms from G15, G16 and A18 as well as oxygens from G15, G16 and G20. This electronegative pocket accommodates the guanidinium moiety of the ligand. The interaction between the guanidine and the receptor is mediated through a hydrogen bond network, as well a specific ionic solvation interaction above and under the guanidinium plane with O6 of G16 and G20. These interactions are well accounted for in the score and docking reproduces this binding geometry. The position of aliphatic portion of the ligand is less well-defined both in the experimental structure and the docking.

### Example 2

### Amp Aptamer

The binding of adenosine monophosphate (amp) is dominated by the stacking of the ligand adenine between 2 purines (A5 and G6). The 6-membered ring of the Adenine is perfectly stacked against the 6 membered ring of the A5 and 5 membered ring of G6. The interaction is stabilised by hydrogen bonds between Adenine N13 and the amino groups of A7 and G12; a hetero purine base pairing of the ligand with G3 further stabilises the complex's (H-bond between G:H21/Adenine:N16 and Adenine:H19/G3:N3.)

There are also several hydrogen bonds between the ligand ribose and RNA backbone and an ionic interaction between the AMP phosphate and the H1 of G6.

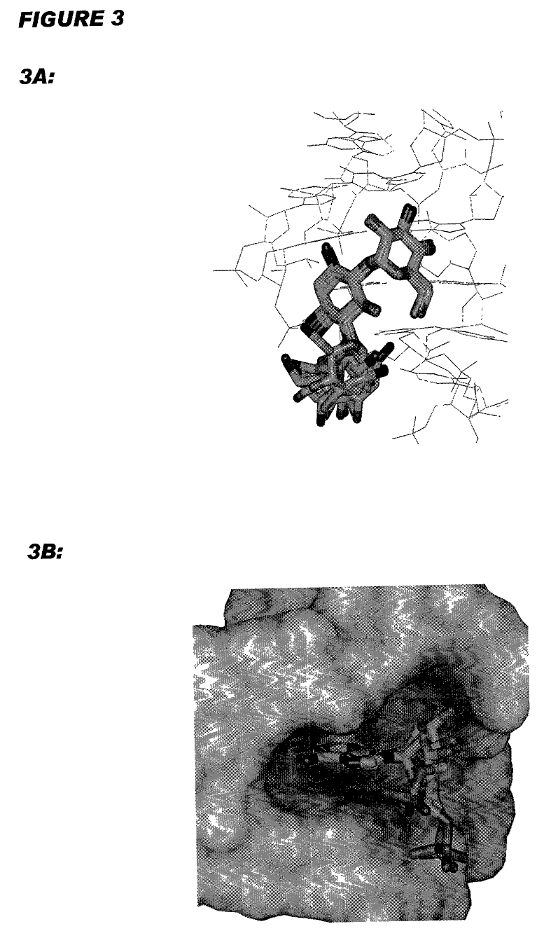

As shown in FIG. 3, the docking is able to reproduce the bound conformation of the ligand adenine moiety. The placement of the phosphate tail is less precise, consistent with the experimental data.

### Example 3

### FMN Aptamer

The flavin Mononucleotide (FMN) aptamer intercalates G10 and G9. The lower plateau of the binding site is formed by G10, U12 and A25 (base triple). The stacking interactions take place between the benzene ring of FMN and 6-membered rings of G10 and G9, and to a lesser extent between the last cycle of FMN and U12. The FMN edge base pairs with A26, displaying 2 hydrogen bonds. The tail also makes hydrogen bonding contacts with bases and the RNA backbone.

The docking predicts correctly the position and orientation of the FMN. Stacking interactions are predicted accurately and so is the hydrogen bond between FMN:O43 and A26:H62.

### Example 4

### Citruline Aptamer

Citruline is an uncharged molecule at physiological pH. Accordingly, in the citruline aptamer, the binding cavity is less negatively charged than what is observed for the arg aptamer. At position 31, G is replaced by U, losing a negatively charged N7. Further, by going from a C at position 13 to a U, N3 is switched from an H-bond acceptor to an H-bond donor, interacting with citruline's urea moiety.

This is the only test-case where the docking method of the invention was not able to predict the binding mode. The authors of citruline aptamer structure note that given the proximity of an array of 4 oxygens from the 4 Gs in the binding cavity, it is likely that a Na⁺ ion may bind this cavity and stabilise the structure. The omission of this ion in the structure and in the docking calculations may thus explain the scoring function's failure.

### Example 5

### Tobramycin Aptamer

The tobramycin ligand in complex with its aptamer show many complementary features involving van der Waals interactions, hydrogen bonding and ionic contacts between the ligand and its receptor, consistent with its tight affinity.

Tobramycin ring I is sandwiched between the major groove and G12 acting as a flap. Amino N8 interacts with U16-O4, U4-O4 and G12-O6. O9H hydrogen bonds with H61 of A15 amine and OH14 hydrogen bonds with G12-O5′. Ring II has ionic interactions between its amine N24 and G5-O1P and G6-N7, as well as between amine N25 and U7-O1P. Finally OH52 hydrogen bonds with the G11 O2P backbone and amine N42 interacts with G12-O1P, U9-O4, and U8-O4.

Docking reproduces the position of ring I and II, scoring for all the above-mentioned interactions.(see FIGS. 3A and 3B). Ring III is slightly less well defined, but the ionic interaction of NH3 is preserved.

Cross-docking

The ability of the scoring function to match ligand and receptor correctly was assessed by cross-docking as described above.

For the four aptamers which were successfully docked (Arg, AMP, fmn, and tobramycin), the correct pairings were largely identified. The pairing for the other aptamer (citrulline) was also acceptable.

Table 3 shows the results, with the diagonal (indicated in bold) corresponding to the docked ΔG values from Table 1.

TABLE 3
Cross-Docking Results

| | ΔG_{bind} | | | | | |
|---|---|---|---|---|---|---|
| | (expt) | | | | | |
| | kJmol⁻¹ | Arg | Amp | fmn | Tob | Cit |
| 1koc (arg) | −28.5 | −31.4 | −17.9 | −21.0 | −23.0 | −20.5 |
| 1am0 (amp) | −28.5 | −26.4 | −32.2 | −21.2 | −21.5 | −21.6 |
| 1fmn (fmn) | −30.1 | −23.1 | −21.4 | −37.4 | −24.0 | −21.6 |
| 2tob (tob) | −45.2 | −32.7 | −20.1 | −28.7 | −38.9 | −24.4 |
| 1kod (cit) | −28.5 | −24.7 | −16.6 | −14.1 | −22.2 | −21.0 |

Tob = tobramycin;
Cit = citrulline

The scoring function, thus, does not allow charged ligands such as arginine and tobramycin to indiscriminately bind all RNA receptors to the same extent. Instead, specific polar contacts and shape complementarity are required.

As shown in the preceding examples, the scoring function according to the invention can predict the correct free energy of binding of a ligand to a receptor, such as an RNA molecule, in most cases. Furthermore, it can discriminate between native and non-native ligands.

The accuracy of the docking method of the invention is also confirmed, and its computational speed indicates that it is suitable for screening large virtual libraries of compounds.

Variations, modifications, and other implementations of what is described herein will occur to those of ordinary skill in the art without departing from the spirit and scope of the invention as claimed. Accordingly, the invention is to be defined not by the preceding illustrative description but instead by the spirit and scope of the following claims.

What is claimed is:

1. A method for determining the free energy of a ligand:RNA complex comprising scoring the pseudo-energy values of one or more molecular interactions between the ligand and RNA molecule.

2. The method of claim 1, wherein said one or more interactions is selected from the group consisting of: hydrogen bonds, lipophilic interactions, ionic interactions, repulsive ionic interactions, aromatic/cation-pi interactions, and entropic costs of interaction.

3. The method of claim 1, wherein formal charges are assigned to receptor and/or ligand atoms prior to scoring.

4. The method of claim 1, wherein said pseudo-energy values are weighted.

5. The method of claim 1, wherein the pseudo-energy value for a hydrogen bond is a function of the donor-acceptor bond length, the acute angle around the donor hydrogen, and the acute angle around the acceptor atom.

6. The method of claim 2, wherein scoring the pseudo-energy value for aromatic/cation-pi interactions comprises determining the average perpendicular distance from each ring center to the other ring plane and the average slip angle between the rings.

7. The method of claim 1, wherein the pseudo-energy value for lipophilic interactions is a linear version of a Lennard-Jones potential function.

8. The method of claim 7, wherein the function has an attractive part and a repulsive part.

9. A method for identifying a putative binding site cavity in a receptor, comprising the following steps:
(a) placing a grid comprising a plurality of grid points over a three-dimensional representation of the receptor;
(b) identifying as an excluded volume, one or more grid points which lie within the van der Waals surface of the receptor;
(c) centering a large sphere over a grid point and determining whether one or more grid points within the large sphere overlaps with the grid points within the excluded volume of the receptor;
(d) removing any grid points which do not overlap;
(e) centering a small sphere over all remaining grid points; determining whether one or more grid points within the small sphere overlap with the excluded volume of the receptor and identifying any non-overlapping grid points as representing a putative binding site cavity within the receptor.

10. The method of claim 9, further comprising the step of dividing grid points identified in step (d) into contiguous cavity regions.

11. The method of claim 9 or 10, further comprising filtering the cavities to remove cavities smaller than a selected minimum size.

12. The method of claim 9 or 11, further comprising the step of eliminating cavities having a center of mass further than a maximum distance from a designated active site center; and identifying any remaining cavities as the binding site of the receptor for the ligand.

13. The method of claim 9, wherein the receptor is a protein, a nucleic acid, a carbohydrate, or a lipid.

14. The method of claim 13, wherein the nucleic acid is RNA.

15. The method of claim 9, wherein the grid is a cube.

16. The method of claim 15, wherein, the spacing between corners of the grid is 0.5 Å.

17. The method of claim 9, wherein the radius of the large sphere is in the range of 3-5 Å.

18. The method of claim 9, wherein the radius of the small sphere is 1.75 Å.

19. The method of claim 11, wherein the minimum size used in step (6) is around 20 grid spacings.

20. The method of claim 12, wherein the maximum distance is 10 Å.

21. A method for docking a ligand to an RNA molecule, wherein the free energy of a ligand:RNA complex's structure is calculated using a scoring function for calculating pseudo-energy values of molecular interactions between the ligand and the RNA molecule.

22. The method of claim 21, wherein a binding site in the RNA molecule to which the ligand binds is identified using the method of claim 9.

23. The method of claim 21, wherein a plurality of ligands are docked to a plurality of RNA molecules.

24. The method of claim 21, said method of docking comprises using Monte Carlo simulated annealing.

25. The method of claim 22, wherein distance restraints are used to keep ligand atoms within a specified distance of at least one of the binding site grid points.

26. The method of claim 25, wherein the specified distance is around 7 Å.

27. A method of screening a library of ligand structures for to identify a ligand which interacts with an RNA molecule, the method comprising: docking a structure from the library against the RNA receptor, to simulate a ligand:RNA complex and calculating the free energy of the ligand:RNA complex's structure using a scoring function for calculating pseudo-energy values of one or more interactions between the ligand and the RNA molecule.
